(19) United States
(12) Patent Application Publication
(10) Pub. No.: US 2024/0182529 A1
(43) Pub. Date: Jun. 6, 2024

(54) PH LOW INSERTION PEPTIDE AND COMPOSITION THEREOF

(71) Applicant: BEIJING ZEQIN BIOMEDICAL CO., LTD, Tongzhou District Beijing (CN)

(72) Inventors: Huawei WEI, Tongzhou District Beijing (CN); Chenggang YANG, Tongzhou District Beijing (CN)

(73) Assignee: BEIJING ZEQIN BIOMEDICAL CO., LTD, Tongzhou District Beijing (CN)

(21) Appl. No.: 16/955,653

(22) PCT Filed: Nov. 30, 2018
(86) PCT No.: PCT/CN2018/118611

§ 371 Date: Jun. 18, 2020

(30) Foreign Application Priority Data
Dec. 19, 2017 (CN) 201711377076.6
Dec. 28, 2017 (CN) 201711464764.6

(51) Int. Cl. C07K 14/195; A61P 35/00; C07K 16/12; A61K 39/00

(52) U.S. Cl. CPC C07K 14/195; A61P 35/00; C07K 16/12; A61K 2039/505

(57) ABSTRACT

An improved pH low insertion peptide which is obtained by repeating the sequence of the extracellular domain of a pH low insertion peptide one or more times based on the sequence of the pH low insertion peptide already existing in the prior art. The pH low insertion peptide uses tumor cells cultured in vitro to prove that the improved pH low insertion peptide is targeted to the surface of tumor cells. Also disclosed are compositions composed of the pH low insertion peptide or the improved type thereof, and these compositions can be used for tumor treatment, diagnosis and identification. The experiments also find that the extracellular domain region of the pH low insertion peptide or the improved type thereof has antigenicity, and the immunized antibody can be used for tumor treatment. The above research results provide a basis for the development of tumor therapeutic drugs.

### TECHNICAL FIELD

The present invention belongs to the field of biomedicine, and relates to a pH low insertion peptide and a composition thereof, and also relates to the role of the composition in targeted treatment of tumors.

### BACKGROUND ART

In recent years, with the rapid development of Chinese economy, the people's material and cultural level has continued to improve, and lifestyles have also undergone tremendous changes, along with changes in the living environment, such as the deterioration of water quality and the decline in air quality. Due to changes in lifestyle and the decline in environmental quality, the causes of death in China have changed greatly, and non-communicable diseases such as malignant tumors, cardiovascular diseases and chronic diseases have become the main causes of death for Chinese residents, wherein deaths caused by malignant tumors account for a large proportion and have become a problem that we cannot ignore.

In the past 10 years, chemotherapy has played a significantly important role in tumor treatment and has also received widespread attention. However, traditional anti-tumor drugs still have many limitations, for example, they cannot distinguish normal tissues from tumor tissues, which makes the treatment efficiency very low, and even worse, it can cause fatal adverse reactions. Therefore, improving selectivity has become the key to the development of anti-tumor drugs. Targeted drug delivery system can specifically deliver anti-tumor drugs to tumor tissues, and can reduce the uptake of anti-tumor drugs in normal tissues, can reduce adverse reactions and improve clinical treatment effects. At present, there are many types of targeted drug delivery systems, and some have been used in clinical treatment. However, the same receptors are expressed in normal tissues, and they can also recognize the targeting ligand, but the recognition level is low, which makes their targeting efficiency and therapeutic effect significantly limited.

The biggest difference between tumor tissues and normal tissues is that the extracellular environment of the former is slightly acidic. In recent years, anti-tumor drugs targeting the acidic microenvironment of tumor tissues have developed rapidly. Due to the high uptake of glucose by tumor cells, glucose is digested into lactic acid under anaerobic conditions, forming an acidic environment; on the other hand, abnormal blood vessels of the tumor cause insufficient oxygen supply to the tumor, and the uncontrolled growth of tumor cell transformation causes hypoxia and metabolic disorders increase anaerobic metabolism; the tumor cells themselves adapt to the hypoxic environment and the acidic environment after the corresponding glycolysis produces lactic acid by up-regulating the hypoxia-inducible factor, which ultimately leads to a pH value of 5.7 to 7.0 in the tumor tissues microenvironment, which is significantly lower than a pH value of 7.4 in the normal tissues. The acidic microenvironment is a very effective target for improving the selectivity of anti-tumor drugs.

The pH low insertion peptide (pHLIP) derived from the transmembrane helix protein C of bacteriorhodopsin has become a research hotspot in recent years because of its special properties in acidic microenvironment. pHLIP is a water-soluble polypeptide which can be inserted into the bilayer lipid membrane of the cell to form a stable transmembrane alpha helix. Peptide folding and membrane insertion are driven by the decline of neutral or alkaline (pH>7.4) pH to weakly acidic (pH=7.0-6.5 or lower). pHLIP has three main forms: form I which has no structure and is dissolved in water at neutral pH, form II which has no structure and is bound to the surface of the cell membrane at neutral pH, and form III which is inserted at acidic pH and passes through the cell membrane with an alpha helix. Because poor solubility due to easy agglutination is the property of membrane peptides, pHLIP as a membrane peptide also has a tendency to agglomerate, especially under the conditions of high concentration and/or low pH. In an aqueous solution of neutral pH, the concentration of the pHLIP monomer is less than 30 μg/ml, and under the condition of low pH, pHLIP of form II and form III are all present in the form of monomers. Many studies have shown that the decrease in peptide solubility due to structural changes can lead to changes in the binding ability of the peptide to the membrane and the conformation of the entire peptide. The stability of peptides in blood is a very important property, because proteases in blood can degrade the peptides composed of L-type amino acids within a few minutes. Although the peptides composed of D-type amino acids are relatively stable, they are not suitable for binding to specific receptors due to their variable chirality. Because there is no specific interaction between pHLIP and lipid bilayers, it is not surprising that pHLIP composed of L-type or D-type has the same biophysical and tumor localization characteristics, and more and more evidence indicates that pHLIP localization does not require any occurrence of specific molecular binding events. The only significant difference is that D-pHLIP forms a transmembrane left-handed helix, while L-pHLIP forms a transmembrane right-handed helix. Compared with cell-penetrating peptides, pHLIP stays in the cell membrane after insertion into the cell membrane, with one end entering the cytoplasm and the other end entering the extracellular space. Therefore, peptide has dual delivery capabilities: one is that it can attach cargo molecules to the surface of cells, and the other is that it can inject or release cargo molecules that cannot penetrate the membrane into the cytoplasm. In order to achieve the first capability, the cargo molecules can be connected to the N-terminus of pHLIP, and such cargo molecules have a wide range of polarities and sizes, and an example of application is the delivery of an imaging probe to acidic tissues and attach it stably to the surface of the cell membrane. In order to achieve the second capability, the cargo molecules can be connected to the C-terminus of pHLIP via a bond that can be cut in the cytoplasm, and such bond that can be cut is disulfide bond, and an example of application is the delivery of anti-tumor drugs to tumor tissues and introduction of them into the tumor cytoplasm to play a role, such as fluorescent dyes, cyclic peptides, polar toxins and peptide nucleic acids.

With the continuous deepening of research on pHLIP, it is found that the application of wild-type pHLIP is limited by some key factors, such as the effect of slow clearance in the body and the charge at the carboxyl end on the membrane insertion process. Scholars try to design pHLIP derivatives with better performance by adjusting the amino acid sequence of pHLIP. The current pHLIP sequence adjustment methods mainly comprise: \textcircled{1} cutting or reversing the membrane insertion end of the wild-type pHLIP sequence; \textcircled{2} replacing part or all of aspartic acid in the transmembrane region with glutamic acid residues, positively charged lysine residues or protonated non-standard amino acid residues (γ-carboxy acid, α-aminooxalic acid). pHLIP derivatives produced by sequence adjustment, such as pHLIP variant 3 (cutting membrane insertion end), can reduce the charge carried by pHLIP, accelerate the process of pHLIP entering the cell membrane to form a transmembrane helix, and improve its tumor targeting. The pHLIP variant 7 accelerates the elimination rate in the blood while maintaining good targeting, which is beneficial for the in vivo delivery of drugs. The current sequence adjustment method of pHLIP can be used to develop more advanced pHLIP derivatives.

### SUMMARY OF THE INVENTION

The present invention provides an improved pH low insertion peptide which comprises the following sequences: polypeptide sequences obtained by repeating the extracellular domain of the WT pH low insertion peptide or a variant thereof once, twice or more times.

Preferably, variants of the WT pH low insertion peptide comprise Var1 to Var16.

The sequences of the WT pH low insertion peptide or the variants thereof are as follows:

WT:
(SEQ ID NO. 1)
ACEQNPIYWARYADWLFTTPLLLLDLALLVDADEGT;
Var 1:
(SEQ ID NO. 2)
ACEDQNPYWARYADWLFTTPLLLLDLALLVDG;
Var 2:
(SEQ ID NO. 3)
ACEDQNPYWRAYADLFTPLTLLDLLALWDG;
Var 3:
(SEQ ID NO. 4)
ACDDQNPWRAYLDLLFPTDTLLLDLLW;
Var 4:
(SEQ ID NO. 5)
ACEEQNPWRAYLELLFPTETLLLELLW;
Var 5:
(SEQ ID NO. 6)
ACDDQNPWARYLDWLFPTDTLLLDL;
Var 6:
(SEQ ID NO. 7)
CDNNNPWRAYLDLLFPTDTLLLDW;
Var 7:
(SEQ ID NO. 8)
ACEEQNPWARYLEWLFPTETLLLEL;
Var 8:
(SEQ ID NO. 9)
CEEQQPWAQYLELLFPTETLLLEW;
Var 9:
(SEQ ID NO. 10)
CEEQQPWRAYLELLFPTETLLLEW;
Var 10:
(SEQ ID NO. 11)
ACEDQNPWARYADWLFPTTLLLLD;
Var 11:
(SEQ ID NO. 12)
ACEEQNPWARYAEWLFPTTLLLLE;
Var 12:
(SEQ ID NO. 13)
ACEDQNPWARYADLLFPTTLAW;
Var 13:
(SEQ ID NO. 14)
ACEEQNPWARYAELLFPTTLAW;
Var 14:
(SEQ ID NO. 15)
TEDADVLLALDLLLLPTTFLWDAYRAWYPNQECA;
Var 15:
(SEQ ID NO. 16)
CDDDDDNPNYWARYANWLFTTPLLLLNGALLVEAEET;
Var 16:
(SEQ ID NO. 17)
CDDDDDNPNYWARYAPWLFTTPLLLLPGALLVEAEET;

The underlined parts of the above sequences are the sequences of the extracellular domains of the pH low insertion peptide. Var1 to Var 16 are all variants of the WT.

The polypeptide sequences obtained by repeating the extracellular domains of the pH low insertion peptide having the sequences of SEQ ID NO. 1 to 17 once, twice or more times comprise:
(extracellular domain)ₙ+linker+SEQ ID NO. 1 to 17, where n=1, 2, 3, 4, etc.

The sequence of the above linker that can be used in the present invention may be (GGGS)ₘ, where m=1, 2, 3, 4, etc.

In a specific embodiment of the present invention, the sequence of the improved pH low insertion peptide is a sequence obtained by repeating the extracellular domain of the Var7 having the sequence of SEQ ID NO. 8, and the sequence is:

(SEQ ID NO. 18)
ACEEQNPGGGSACEEQNPWARYLEWLFPTETLLLEL.

In the specific embodiments of the present invention, although the Var7 is taken as an example, it is proved that the sequence obtained by repeating the extracellular domain of the pH low insertion peptide once has a more beneficial effect than the original sequence, and those skilled in the art will directly and unequivocally conclude that for other variants of the WT, the sequences obtained by repeating their extracellular domains once can also have more beneficial effects than the original sequence. Because the experimental results of the present invention indicate the commonality of the advantages of the extracellular domains of the pH low insertion peptide, the modified pH low insertion peptides of the above WT and WT variants comprising Var1 to Var 16 are included in the protection scope of the present invention.

The present invention provides a composition, which comprises the aforementioned improved pH low insertion peptide, or the pH low insertion peptide having the sequence of SEQ ID NO. 1 or the variant thereof.

Further, wherein the composition comprises a functional body which comprises therapeutic agents, diagnostic agents, and marker molecules.

The functional body is connected to the N-terminus or C-terminus of the aforementioned improved pH low insertion peptide, or to the N-terminus or C-terminus of the aforementioned pH low insertion peptide having the sequence of SEQ ID NO. 1 or the variant thereof.

Specifically, if a therapeutic agent exerts a therapeutic effect through molecules on the surface of the cell, the therapeutic agent requires to be connected to the N-terminus of the pH low insertion peptide, and if a therapeutic agent exerts a therapeutic effect through molecules inside the cell, the therapeutic agent requires to be connected to the C-terminus of the pH low insertion peptide, The diagnostic agent is used to show the existence of pathological state of diseases, which can be connected to the N terminus to display on the cell surface, or can be connected to the C terminus to display in the cytoplasm; The marker molecule is used to increase the expression of the marker molecule on the surface of the cell membrane not comprising the marker molecule, therefore, in general, the marker molecule is connected to the N-terminus of the pH low insertion peptide.

Further, the therapeutic agents include, but are not limited to, antibody drugs, small molecule drugs, antibiotics, polypeptides, peptide nucleic acids, nanoparticles, and liposomes.

The antibody drugs may be antibody drugs directed against any tumor molecule, as long as they can treat the tumor. Antibody drugs comprise: molecular targeted monoclonal antibody drugs, targeted antibody-conjugated drugs, bispecific antibody drugs, targeted immune checkpoint drugs, etc. Examples of such antibody drugs include, but are not limited to: rituximab, trastuzumab, gemtuzumab, alemtuzumab, ibritumomab, tositumomab, bevacizumab, cetuximab, panitumumab, ofatumumab, denosumab, epimumumab, bentuximab, pertuzumab, ado-trastuzumab, obinutuzumab, ramucirumab, pembrolizumab, blinatumomab, nivolumab, daratumumab, dinutuximab, necitumumab, elotuzumab, atezolizumab, avelumab, denosumab, gemtuzumab, necitumumab, and atezolizumab.

Further, the antibiotics comprise anti-tumor antibiotics, which are chemical substances with anti-tumor activity produced by microbial metabolism. Anti-tumor antibiotics that can be used in the present invention comprise: C1027, mitomycin, adriamycin, CC-1065, adozelesin, ducarmycins, gilvusmycin, tetracyclines, cinnamamide, MMI-166, batimastat, green tea polyphenol, salvianolic acid A, C3368-A, C3368-B, emodin, tricyclic pyrones, gel danamycin, 17AAG, paclitaxel, epothilone A, epothilone B, calicheamicin, lidamycin.

Further, the small molecule drugs are usually signal transduction inhibitors, which can specifically block the signal transduction pathways necessary for tumor growth and proliferation to achieve the purpose of treatment, and examples of the small molecule drugs include, but are not limited to: imatinib, nilotinib, dasatinib, everolimus, erlotinib, sunitinib, ibrutinib, sorafenib, crizotinib, pazopanib, gefitinib, carfilzomib, tofacitinib, axitinib, regorafenib, vemurafenib, sirolimus, ponatinib, levatinib, olapanib, aflibercept, ceritinib, romidepsin, alectinib, belincstat, bosutinib, vandetanib, cabozantinib, panobinostat, afatinib, palifermin, trametinib, dabrafenib, temsirolimus, lapatinib, vorinostat, venetoclax, gleevec, iressa.

Further, examples of the polypeptide include, but are not limited to, toxins, cyclic peptides, microtubule inhibitors, protease activated receptors. An example of the toxins is amanitin, an example of the cyclic peptidesis phalloidin, an example of the microtubule inhibitors is monomethyl auristatin E (MMAE), and an example of the protease activated receptors is P1AP.

Further, the peptide nucleic acids comprise anti-miR (antisense nucleic acid) oligonucleotide peptides.

Further, the nanoparticles comprise: chitosan targeted nanoparticles, long circulation nanoparticles, polylactic acid nanoparticles, solid lipid nanoparticles, gold nanoparticles, mesoporous silicon nanoparticles loaded with doxorubicin, and superparamagnetic iron oxide nanoparticles.

Further, the liposomes comprise phospholipids and cholesterol.

The phospholipids of the present invention include, but are not limited to, one or more of: soybean phosphatidylcholine (SPC), polyethylene glycol 1000 vitamin E succinate (TPGS), dimyristoyl phosphatidylcholine (DMPC), dilauroyl lecithin (DLPC), distearoyl phosphatidylcholine (DPPC), dipalmitoyl phosphatidylcholine (DPPC), distearoyl phosphatidylcholine (DSPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1-stearoyl-2-palmitoyl phosphatidylcholine (SPPC), egg phosphatidylcholine (EPC), hydrogenated soybean phosphatidylcholine (HSPC), dioleoyl phosphatidylcholine (DOPC), dioleoyl phosphatidylethanolamine (DOPE), dilauroyl phosphatidylglycerol (DLPG), dipalmitoyl glycerol (DPPG), distearoyl plhosphatidylglycerole (DSPG), dioleoyl phosphatidylglycerol (DOPG), dimyristoyl phosphatidic acid (DMPA), dipalmitoyl phosphatidic acid (DPPA), dimyristoyl phosphatidylethanolamine (DMPE), dipalmitoyl phosphatidylethanolamine (DPPE), dimyristoyl phosphatidylserine (DMPS), dipalmitoyl phosphatidylserine (DPPS), cerebral phosphatidylserine (PS), cerebral sphingomyelin (BSP), dipalmitoyl sphingomyelin (DPSP), distearoyl sphingomyelin (DSSP), and distearoyl phosphatidylethanolamine (DSPE), wherein the preferred are: soybean phosphatidylcholine (SPC), distearoyl phosphatidylethanolamine (DSPE) or dioleoyl phosphatidylethanolamine (DOPE).

Further, the diagnostic agents comprise fluorescent dyes. Fluorescent dyes include, but are not limited to, Alexa750, Alexa546, Alexa647, Cy5.5, DyLight 680, DyLight 680 4* PEG-conjugate (DyP680), IRDye® 680RD (IR680), IRDye® 800CW (IR800), indocyanine green ICG, PE, Percy-Cy5.5, FITC, APC, Cy7, FITC, GFP, Alexa Fluar488, Bidipy, Fluo-3, Propidium Iodide (PI), PerCP, PE-Cy5, PE-Teses Red, 7-AAD, PE-Cy7, PE-Alexa Flour750, Alexa Fluor660, Alexa Fluor700, APC-Cy7, APC-Alexa Flour750, Hoechsr33342-Blue, DAPI, Hoechsr33342-Red, arific Blue, Cascade Blue, Alexa Flour 405, and Parific orange.

Further, the marker molecule comprises tumor surface antigens or functional domains thereof, and the tumor surface antigens generally refer to newly appeared or overexpressed antigen substances on the cell surface during occurrence and development of tumors.

Examples of tumor surface antigens include, but are not limited to, ER, PR, P53, EGFR, IGFR, Her2, CD20, CD25, CD117, CD34, CD138, CD33, VEGFR, BCMA, Mesothelin, CEA, PSCA, MUC1, EpCAM, S100, CD22, CD19, CD70, CD30, ALK, RANK, GPC2, GPC3, Her3, EGFRvIII, GD2, PD-L1, and PD-L2.

The aforementioned marker molecules of the present invention are connected to the N-terminus of the aforementioned improved pH low insertion peptide, or to the N-terminus of the aforementioned pH low insertion peptide having the sequence of SEQ ID NO. 1 or the variant thereof via a linker.

Further, the aforementioned marker molecules are connected to the N terminus of the pH low insertion peptide having the sequence of SEQ ID NO. 1 via a linker.

The above-mentioned Linker is conventionally used in the art, and the sequence of the linker is (GGGS) m or (GGGGS) m, where m=natural number.

In a specific embodiment of the present invention, the sequence of the linker is GGGGS (SEQ ID NO. 19).

In a specific embodiment of the present invention, the marker molecules are tumor surface antigen Her2 or functional domains thereof.

Further, the functional domain of the tumor surface antigen Her2 is a fourth domain of an Her2 protein or a functionally similar domain thereof, or a second domain of the Her2 protein or a functionally similar domain thereof; the sequence of the fourth domain of the Her2 protein is shown in SEQ ID NO. 20, the functionally similar domain of the fourth domain of the Her2 protein is a polypeptide derived from the amino acid as shown in SEQ ID NO. 20 and retaining the antibody binding activity of the fourth domain of the Her2 protein, and the sequence of the second domain of the Her2 protein is shown in SEQ ID NO. 23, and the functionally similar domain of the second domain of the Her2 protein is a polypeptide derived from the amino acid as shown in SEQ ID NO. 23 and retaining the antibody binding activity of the second domain of the Her2 protein.

Further, the functional domain of the tumor surface antigen Her2 is a fourth domain of an Her2 protein or a functionally similar domain thereof, and the functionally similar domain of the fourth domain of the Her2 protein retains the antibody binding activity of the fourth domain of the Her2 protein.

The functionally similar domains of the fourth domain of the Her2 protein comprise polypeptides derived from the amino acid sequence as shown in SEQ ID NO. 20, which is obtained by substitution and/or deletion and/or addition of one or several amino acid residues from the fourth domain of the Her2 protein having the sequence of SEQ ID NO. 20, and has the same function as the sequence as shown in SEQ ID NO. 20.

The functionally similar domains of the fourth domain of the Her2 protein comprise polypeptides composed of the amino acid sequences which have at least 80% homology (also known as sequence identity) to the amino acid sequence as shown in SEQ ID NO. 20, more preferably, have at least about 90% to 95% homology, and often 96%, 97%, 98%, and 99% homology with the amino acid sequence as shown in SEQ ID NO. 20.

The functionally similar domains of the second domain of the Her2 protein comprise polypeptides derived from the amino acid sequence as shown in SEQ ID NO. 23, which is obtained by substitution and/or deletion and/or addition of one or several amino acid residues from the second domain of the Her2 protein having the sequence of SEQ ID NO. 23, and has the same function as the sequence as shown in SEQ ID NO. 23.

The functionally similar domains of the second domain of the Her2 protein comprise polypeptides composed of the amino acid sequences which have at least 80% homology (also known as sequence identity) to the amino acid sequence as shown in SEQ ID NO. 23, more preferably, have at least 90% to 95%, and often 96%, 97%, 98%, and 99% homology with the amino acid sequence as shown in SEQ ID NO. 23.

Generally, it is known that modifications of one or more amino acids in a protein or polypeptide do not affect the function of the protein. Those skilled in the art will approve that changing a single amino acid or a small percentage of amino acids or individual additions, deletions, insertions, and substitutions to amino acid sequences are conservative modifications, where changes in protein polypeptides generate proteins or polypeptides with similar functions. Conservative substitution tables that provide functionally similar amino acids are well known in the art.

The functionally similar domains of the fourth domain of the Her2 protein also comprise non-conservative modifications to the amino acid sequence as shown in SEQ ID NO. 20, as long as the modified polypeptide still retains the biological activity of the bound antibody.

The functionally similar domains of the second domain of the Her2 protein also comprise non-conservative modifications to the amino acid sequence as shown in SEQ ID NO. 23, as long as the modified polypeptide still retains the biological activity of the bound antibody.

Preferably, the functional domain of the tumor surface antigen Her2 is the fourth domain of the Her2 protein with the sequence shown in SEQ ID NO. 20; or the second domain of the Her2 protein with the sequence shown in SEQ ID NO. 23.

In a specific embodiment of the present invention, the fourth domain of the Her2 protein is connected to the N-terminus of the pH low insertion peptide having the sequence of SEQ ID NO. 1 via a linker, the sequence of the linker is GGGGS, and the sequence of the composition is shown in SEQ ID NO. 21.

In a specific embodiment of the present invention, the second domain of the Her2 protein is connected to the N-terminus of the pH low insertion peptide having the sequence of SEQ ID NO. 1 via a linker, the sequence of the linker is GGGGS, and the sequence of the composition is shown in SEQ ID NO. 24.

The present invention also provides a neoantigen, and the neoantigen sequence comprises the extracellular domain sequence of the aforementioned improved pH low insertion peptide or a variant sequence thereof, or the extracellular domain sequence of the aforementioned pH low insertion peptide shown in SEQ ID NO. 1 or a variant sequence thereof.

In a specific embodiment of the present invention, the neoantigen sequence is shown in SEQ ID NO. 22.

The neoantigen of the present invention has the following functions: (1) antigenicity; (2) connected with carrier protein as an immunogen to stimulate animals to produce a specific antibody.

The preparation method of the neoantigen of the present invention can use the chemical synthesis method: using an automatic polypeptide synthesizer to synthesize the antigen by the solid phase method.

The present invention also provides a nucleic acid molecule, which encodes the aforementioned neoantigen.

The present invention also provides a recombinant vector, which is composed of an empty vector and a target gene inserted into the empty vector, and the target gene is the aforementioned nucleic acid molecule.

In the present invention, the “empty vector” (or called “vector”) can be selected from various vectors known in the art, such as various commercially available plasmids, cosmids, phages, and retroviruses. The empty vector may comprise a variety of commonly used detection markers (reporter genes such as fluorescent markers and antibiotic markers) and restriction sites. Recombinant vectors can be constructed by using various endonucleases from the multiple cloning sites of the empty vector to digest to obtain linear plasmids, which are connected to gene fragments digested with the same endonucleases to obtain recombinant plasmids.

The present invention also provides a recombinant host cell, which includes the aforementioned recombinant vectors.

The recombinant vectors can be transformed, transduced or transfected into host cells by conventional methods in the art, such as calcium chloride chemical transformation, high-voltage electroporation, and preferably electroporation; the host cells may be prokaryotic cells or eukaryotic cells, preferably E. coli, Bacillus subtilis, yeast (e.g., Pichia pastoris) or various animal and plant cells, and more preferably the host cells are genetically engineered bacteria commonly used in the art such as E. coli, Bacillus subtilis or Pichia pastoris.

The neoantigen of the present invention can be isolated and purified from the recombinant host cells using methods commonly used in the art. For example, centrifugal separation of culture medium and recombinant host cells, high-pressure homogenization to break cells, centrifugal filtration to remove cell debris, and affinity chromatography to purify the neoantigen. For the resulting neoantigen product by isolation and purification, purity identification can be performed using methods commonly used in the art. For example, coomassie brilliant blue method, kjeldahl method, biuret method, lowry method, ultraviolet absorption method, affinity chromatography, antigen-antibody method, electrophoretic analysis (e.g., sodium dodecyl sulfonate polyacrylamide gel electrophoresis), sedimentation analysis, diffusion analysis, constant solubility method, protein profile, etc.

The present invention also provides a fusion protein, which comprises the aforementioned neoantigen.

Further, the fusion protein comprises the aforementioned neoantigen and a protein or polypeptide connected to the neoantigen.

Furthermore, the fusion protein comprises the aforementioned neoantigen and a carrier protein coupled to the neoantigen.

In a specific embodiment of the present invention, the fusion protein is prepared by coupling the aforementioned neoantigen to a carrier protein.

Carrier proteins that can be used in the present invention include, but are not limited to, KLH (keyhole limpet hemocyanin), bovine serum albumin (BSA), ovalbumin OVA, etc. Since KLH (keyhole limpet hemocyanin) has strong immunogenicity, many binding sites, good immunization effects, and a distant relationship with immunized animals, using it as a carrier protein is not likely to cause cross-reaction, it is preferred.

The fusion protein of the present invention has immunogenicity and specificity and is an immunogen that can be used to immunize animals to prepare specific antibodies against the aforementioned neoantigen.

The present invention also provides a novel antibody prepared from the aforementioned neoantigen or the aforementioned fusion protein.

Preferably, the aforementioned novel antibody of the present invention is a monoclonal antibody.

The monoclonal antibody of the present invention can be prepared using conventional techniques in the art, and methods commonly used in the prior art to prepare antibodies comprise:

### (1) Hybridoma Technique Based on Mouse/Rabbit.

Basic steps: Animal immunity, cell fusion, screening of hybridoma cells and detection of monoclonal antibodies, cloning of hybridoma cells, identification and preparation of monoclonal antibodies, etc.

### (2) Antibody Screening Technique Based on Phage Antibody Display Library.

Basic steps: \textcircled{1} Isolating B lymphocytes from peripheral blood or spleen, lymph nodes and other tissues, extracting mRNA and reverse transcribing to cDNA; \textcircled{2} Amplifying different Ig gene fragments by PCR technology according to the needs of library construction using antibody light chain and heavy chain primers; \textcircled{3} Constructing phage vectors; \textcircled{4} Transforming bacteria with expression vectors to construct a full set of antibody library. Through multiple rounds of antigen affinity adsorption-elution-amplification, antigen-specific antibody clones are finally screened.

### (3) Screening Technique Based on Monoclonal Antibody Library.

The aforementioned pH low insertion peptide in the present invention can be prepared using conventional techniques in the art, and such synthesis techniques comprise: solid phase synthesis, and liquid phase synthesis.

The principle of solid phase synthesis lies in: the carboxyl terminus of the amino acid is fixed on the insoluble resin via an appropriate linking molecule, and then the amino acid is sequentially condensed on the resin after deprotection of the amino group to extend the peptide chain until the desired polypeptide is obtained. Finally, the side chain protecting group is removed with an appropriate reagent and a product is cracked from the resin. Compared with liquid phase, the advantages of polypeptide solid phase synthesis are: (1) each step of the reaction only requires simple filtration and washing of the resin to achieve the purpose of purification, overcoming the difficulty of purifying the product of each step in the classic liquid phase synthesis method, saving time and effort in operation; (2) soluble reagents can be overdose to complete the reaction and obtain a high yield, and excess reagents can be simply rinsed with solvent and removed by filtration; (3) all reactions can be carried out in one container, thus avoiding the procedure and loss of transfer of reaction intermediates; (4) if appropriate linking molecules and cracking conditions are selected, the polymer resin can be recycled and reused.

Strategies of polypeptide solid phase synthesis comprise the Boc solid-phase method and Fmoc solid-phase method. In a specific embodiment of the present invention, the present invention uses the Fmoc solid phase method.

The present invention provides a tumor marking system, which comprises the aforementioned improved pH low insertion peptide, or the pH low insertion peptide having the sequence of SEQ ID NO. 1 or the variant thereof.

The present invention provides a tumor marking system, which comprises the aforementioned composition comprising a marking molecule.

Further, the tumor marking system may further comprise Cyanine 5.5, Alexa Flour 750, Alexa Fluor 647, Alexa Flour 488, Alexa Flour 546, ⁶⁴Cu-DOTA, ⁶⁸Ga-DOTA, ¹⁸F—O-pyridine, ¹⁸F-liposomes, liposomal Rhodamine, Nanogold, and TAMRA.

The construction of the tumor marking system of the present invention is based on the following ideas: Tumors are heterogeneous, even though surface of tumor cells in the same tumor tissue may express different protein antigens, drugs against a certain protein antigen can only kill tumor cells expressing the antigen, but have no killing effect to tumor cells that do not express the antigen, and these tumor cells survive to form a growth advantage, making the tumor patients resistant to the drugs. If a protein antigen is expressed on the surface of all tumor cells, it will cause the drug against the protein antigen to completely kill all tumor cells. The same is true for different tumor tissues. The present invention connects molecular typing markers expressed on the surface of cancer cells with targeted tumor pH low insertion peptide or improved forms thereof to form a composition, which can target any solid tumor cells and display on the surface of tumor cells. Drugs targeting molecular typing markers expressed on the surface of cancer cells, such as the drug trastuzumab against the fourth domain of the Her2, can kill any cancer cells, including breast cancer cells, and expand the scope of application of this tumor drug.

The present invention provides a targeted tumor therapeutic system, which comprises the aforementioned tumor marking system or the aforementioned composition comprising therapeutic agents.

The targeted tumor therapeutic system comprises the aforementioned tumor marking system and tumor killing system which comprises the aforementioned novel antibody, or an antibody to the tumor surface antigen or functional domain thereof.

In detail, the targeted tumor therapeutic system may comprise two sub-systems, one sub-system is a tumor marking system, comprising the aforementioned composition comprising a marking molecule in the present invention, and the other sub-system is a tumor killing system, comprising an antibody to the aforementioned neoantigen, or an antibody against the tumor surface antigen or functional domain thereof.

The antibody to the aforementioned neoantigen and the antibody against the tumor surface antigen of the present invention may be any antibody. The antibodies comprise monoclonal antibodies and bispecific antibodies.

The antibody to the aforementioned neoantigen, or the antibody against the tumor surface antigen of the present invention also comprise antigen-binding parts against antibodies, further, the antigen-binding fragments of the antibodies comprise Fab, Fab′, F(ab′)₂, Fv or single chain antibody.

Fab refers to a part of an antibody molecule that contains a variable region and a constant region of a light chain and a variable region and a constant region of a heavy chain bound by a disulfide bond.

Fab′ refers to a Fab fragment containing part of the hinge region.

F(ab′)₂ refers to the dimer of Fab′.

Fv refers to the smallest antibody fragment that contains a heavy chain variable region and a light chain variable region of the antibody and has all antigen binding sites.

Single chain antibody refers to an engineered antibody composed of a light chain variable region and a heavy chain variable region directly connected or connected by a peptide chain.

The antibody to the aforementioned neoantigen, or the antibody against the tumor surface antigen of the present invention also comprises various variants of antibodies, such as those derived from similar amino acid substitutions well known in the art, and variants caused by amino acid deletions and increases.

The antibody to the aforementioned neoantigen, or the antibody against the tumor surface antigen of the present invention may contain one or more glycosylation sites in the variable regions of the heavy and light chains, as is well known in the art, the presence of one or more glycosylation sites in the variable regions can lead to enhanced antibody immunogenicity, or changes in antibody pharmacokinetics due to altered antigen binding.

The antibody to the aforementioned neoantigen, or the antibody against the tumor surface antigen of the present invention can be designed to contain modifications in the Fc region, usually to change one or more functional properties of the antibody, such as serum half-life, complement binding, Fc receptor binding, and/or antigen-dependent cytotoxicity. In addition, the antibody of the present invention can be chemically modified (e.g., one or more chemical groups can be attached to the antibody), or modified to change its glycosylation, thereby further changing one or more functional properties of the antibody.

Another modification that can be designed of the antibody to the aforementioned neoantigen, or the antibody against the tumor surface antigen of the present invention is pegylation. The antibody can be pegylated to, for example, increase the biological half-life of the antibody (e.g., serum). In order to pegylate an antibody, the antibody or fragments thereof is usually reacted with PEG under conditions suitable for the attachment of one or more polyethylene glycol (PEG) groups to the antibody or antibody fragments, e.g., active esters or aldehyde derivatives of polyethylene glycol. Preferably, the pegylation is achieved by performing an acylation reaction or an alkylation reaction with an active PEG molecule (or similar active water-soluble polymers).

Examples of antibodies against tumor surface antigens include, but are not limited to: molecular targeted monoclonal antibody drugs, targeted antibody-conjugated drugs, bispecific antibody drugs, targeted immune checkpoint drugs, etc. Examples of such antibody drugs include: rituximab, trastuzumab, gemtuzumab, alemtuzumab, ibritumomab, tositumomab, bevacizumab, cetuximab, panitumumab, ofatumumab, denosumab, epimumumab, bentuximab, pertuzumab, ado-trastuzumab, obinutuzumab, ramucirumab, pembrolizumab, blinatumomab, nivolumab, daratumumab, dinutuximab, necitumumab, elotuzumab, atezolizumab, avelumab, denosumab, gemtuzumab, necitumumab, and atezolizumab.

The tumor killing system of the present invention may be a CAR-T or TCR-T system, which expresses antibodies or TCRs against tumor surface antigens through immune cells, such as T cells. The tumor killing system can also be an ADC (antibody drug conjugates) system, i.e., antibody-coupled toxins (Pseudomonas aeruginosa exotoxin PE38, diphtheria toxin, duocarmycin, staphylococcal enterotoxin A/E-120, shiga toxin, ricin,), chemotherapeutic drugs (irinotecan, exatecan, adriamycin), small molecule inhibitors (orestatins, calicheamycins, maytansines, tubulysin, antibacterial drugs, urease), liposomes, gold nanoparticles, etc. In addition, the tumor killing system can also be immunocytokines, which connects certain immune cytokines with antibodies, such as IL-2, IL-12, TNF-α, IL-10, TGF-β, etc. It also comprises a bispecific antibody killing system, i.e., one antibody recognizes an antigen or antigen domains connected by the fusion peptide, and the other antibody recognizes the other antigen.

The functioning principle of the targeted tumor therapeutic system of the present invention is as follows: the marking molecules in the tumor marking system are inserted into the cell membrane under the action of pH low insertion peptide, the marking molecules display on the surface of tumor cells, and antibody drugs in the tumor killing system recognize the marking molecules on the surface of tumor cells, thus concentrating the tumor killing system on the tumor tissue and killing tumor cells thoroughly and specifically.

The present invention also provides the use of the aforementioned improved pH low insertion peptide, or the aforementioned pH low insertion peptide having the sequence of SEQ ID NO. 1 or the variant thereof in the preparation of the aforementioned composition.

Specifically:

The present invention provides the use of the aforementioned improved pH low insertion peptide, or the aforementioned pH low insertion peptide having the sequence of SEQ ID NO. 1 or the variant thereof in the preparation of a tumor drug targeted delivery system. The aforementioned tumor therapeutic agent is connected to a pH low insertion peptide, and depending on the targeting of the pH low insertion peptide to a slightly acidic environment, enabling the targeted delivery of the tumor therapeutic agent to tumor tissues and the specific killing of the tumor tissues.

The present invention provides the use of the aforementioned improved pH low insertion peptide, or the aforementioned pH low insertion peptide having the sequence of SEQ ID NO. 1 or the variant thereof in the preparation of a tumor diagnostic tool. The aforementioned tumor diagnostic agent is connected to a pH low insertion peptide, and depending on the targeting of the pH low insertion peptide to a slightly acidic environment, enabling the targeted delivery of the tumor diagnostic agent to tumor tissues and the marking of the presence of the tumor tissues to determine whether the subject has a tumor.

The present invention provides the use of the aforementioned improved pH low insertion peptide, or the aforementioned pH low insertion peptide having the sequence of SEQ ID NO. 1 or the variant thereof in the preparation of a tumor marking system. The aforementioned tumor surface antigen is connected to a pH low insertion peptide, and depending on the targeting of the pH low insertion peptide to a slightly acidic environment, enabling the targeted delivery of the tumor surface antigen and the stay on the surface of tumor cells, so that the tumor cells are marked by the tumor surface antigen, which is beneficial to tumor drugs targeting the specific antigen to play a killing effect on the tumor. Taking HER2 as an example, trastuzumab only has a therapeutic effect on HER2-positive breast cancer patients, by targeting the HER2 connected by a pH low insertion peptide to the surface of breast cancer cells in the HER2-negative breast cancer patients, trastuzumab can also play a therapeutic role in the HER2-negative breast cancer patients, expanding the application scope of trastuzumab.

The present invention also provides the use of the aforementioned improved pH low insertion peptide, the aforementioned pH low insertion peptide having the sequence of SEQ ID NO. 1 or the variant thereof, the aforementioned composition comprising a therapeutic agent, the aforementioned composition comprising a marking molecule, or the aforementioned tumor marking system in the preparation of the aforementioned targeted tumor therapeutic system.

The present invention also provides the use of the aforementioned composition in the preparation of the aforementioned tumor marking system.

The present invention also provides the use of the aforementioned composition in the preparation of the aforementioned tumor therapeutic system.

The present invention also provides the use of the aforementioned tumor marking system in the preparation of the aforementioned tumor therapeutic system.

The present invention also provides the use of the aforementioned neoantigen in the preparation of the aforementioned fusion protein, the aforementioned novel antibody or the aforementioned tumor killing system.

The present invention also provides the use of the aforementioned novel antibody in the preparation of the composition comprising a therapeutic agent, or the aforementioned tumor killing system.

The present invention also provides the use of the aforementioned improved pH low insertion peptide, or the aforementioned pH low insertion peptide having the sequence of SEQ ID NO. 1 or the variant thereof in the preparation of CAR-T sequence. Antibodies screened and obtained by using the pH low insertion peptide or the improved type thereof in the present invention as an antigen can be used as a completely new Scfv sequence to design CAR-T sequences.

The term “CAR-T” herein refers to chimeric antigen receptor T-cell Immunotherapy. According to the characteristics of the tumor microenvironment, scientists have optimized a series of CART sequences so that they have a completely different affinity for antigens at different pH values and thus activate at different pH values.

The term “tumor surface antigen” herein generally refers to newly appeared or overexpressed antigen substances on the cell surface during occurrence and development of tumors.

The term “targeted antibody-conjugated drug” herein can also be called immunoconjugate. The immunoconjugate molecule is composed of two parts of a monoclonal antibody and a “warhead” drug. There are three main types of substances that can be used as a “warhead”, i.e., radionuclides, drugs and toxins; each of which is connected with the monoclonal antibody to constitute radioimmunoconjugate, chemical immunoconjugate and immunotoxin, respectively.

The term “bispecific antibody drug” herein refers to an antibody that can bind to two epitopes at the same time, and the bispecific antibodies can be divided into two types, i.e., T cell recruitment type, which comprises tumor cell target-T cell recruitment sites and accounts for a majority of bispecific antibodies, where the T cell recruitment sites refer to CD3 (T cells) and CD16 targets (NK cells), which are usually located in tumor cells; in addition, the bispecific antibodies may also bind to double target sites (e.g., VEGF-PDGF, VEGF-Ang2), and inhibit 2 signal pathways, thereby reducing the possibility of drug resistance.

The term “peptide nucleic acid” (PNA) is a synthetic DNA or RNA analogue, and its skeleton is composed of repeated N-2 (aminoethyl)-glycine (N (2-aminoethyl) glycine) units, and the base and the backbone are connected by a methylene carbonyl bond. Since it has no phosphate groups like DNA or RNA, it is electrically neutral, has no electrical repulsion with DNA and RNA, has strong base pairing specificity, can form a stable complex when combined with DNA and RNA, and high stability. In addition, PNA is not easily hydrolyzed by proteases or nucleases, therefore, PNA has broad application prospects in the field of biological research and clinical medicine.

The term “monoclonal antibody” herein refers to a monoclonal antibody produced by a single B cell clone, which is highly uniform and only against a specific epitope.

The polypeptide sequences in the present invention are listed in order from N-terminus to C-terminus.

The advantages and beneficial effects of the present invention: The present invention improves the sequence on the basis of the known pH low insertion peptide, and the improved polypeptide has stronger selectivity in the acidic microenvironment of tumor tissues and maintains in vivo longer.

The present invention, for the first time, connects the fourth domain of the tumor surface antigen Her2 to a pH low insertion peptide to form a composition that can mark tumors. The research achievements of the present invention greatly extend the indications of existing tumor drugs against one cancer or one specific type of cancer, and are of great significance for clinical treatment of tumors.

### BRIEF DESCRIPTION OF THE DRAWINGS

FIG. 1 shows a fluorescence diagram of localization of the pH low insertion peptide var7 on cells;

FIG. 2 shows a fluorescence diagram of localization of the improved pH low insertion peptide p-var7 on cells;

FIG. 3 shows a diagram of studying the localization of pH low insertion peptide in animals using in vivo imaging technique, where A: 24 h; B: 48 h; C: 72 h; D: 72 h, tumor tissues obtained from by dissecting mice;

FIG. 4 shows a graph of the effect of the 1G12 on tumor growth;

FIG. 5 shows a statistical graph of the effect of the 1G12 on tumor weight;

FIG. 6 shows a statistical graph of the effect of the 1G12 on mouse weight;

FIG. 7 shows a pathological staining diagram of the liver;

FIG. 8 shows a pathological staining diagram of the kidney;

FIG. 9 shows a pathological staining diagram of the lung;

FIG. 10 shows a pathological staining diagram of the large intestine;

FIG. 11 shows a pathological staining diagram of the spleen;

FIG. 12 shows a electrophoresis diagram of the expressed and purified fourth domain of the Her2-pHLIP identified by SDS-PAGE;

FIG. 13 shows a fluorescence diagram of the Her2 protein expression in A549 cells observed by cofocal;

FIG. 14 shows a fluorescence diagram of the localization of the fourth domain of the Her2-pHLIP on A549 cells in a neutral environment observed by cofocal;

FIG. 15 shows a fluorescence diagram of the localization of the fourth domain of the Her2-pHLIP on A549 cells in a acidic environment observed by cofocal;

FIG. 16 shows a graph of the fourth domain of the Her2-pHLIP on tumor growth.

### DETAILED DESCRIPTION OF EMBODIMENTS

The content of the present invention can be more easily understood by referring to the following examples which are only for further illustrating the present invention and are not meant to limit the scope of the present invention.

Unless otherwise specified, the experimental methods used in the following examples are conventional methods.

Unless otherwise specified, the materials and reagents used in the following examples can be obtained from commercial sources.

### Example 1 Synthesis of the Improved pH Low Insertion Peptide

According to the sequences of SEQ ID NO. 8 and SEQ ID NO. 18, it was sequentially synthesized from the carboxyl end to the amino end.

### (1) Connection of the First Amino Acid to the Resin

1 g of 2-Chlorotrtyl Chloride Resin was placed in a dry and clean peptide synthesis column, to which 8 ml of DCM was added to swell for 5 min, and the solvent was aspirated and discarded under vacuum. 2 mmol of Fmoc amino acid and 5 mmol of DIEA were taken respectively to dissolve in 8 ml of DCM, and added to the resin to react under gentle shaking for 60 min at room temperature. The solvent was aspirated and discarded under vacuum. The resin was washed twice with 10 ml of DMF for 2 min each time. 10 ml of DCM/methanol/DIEA (80:15:5) was added to react under gentle shaking for 10 min, and the solvent was aspirated and discarded under vacuum. It was repeated once. The resin was washed 3 times with 10 ml of DMF for 2 min each time. The solvent was aspirated and discarded under vacuum, and it was blown dry with N₂.

### (2) Determination of the Coupling Rate of the First Amino Acid to the Resin

2 mg of dried Fmoc amino acid-resin was weighed accurately and placed into a cuvette, to which 3 ml of 20% piperidine/DMF was added to react under gentle shaking for 10 min. 20% piperidine/DMF was used as an empty self-control and adjusted to zero, and the light absorption value of the sample at 290 nm was measured with an ultraviolet spectrophotometer. The measurement was repeated twice and the average value is taken. The coupling rate was calculated by the following formula:

coupling rate (mmol/g)=(Abs sample)/(sample weight mg*1.75)

### (3) Deprotection of the Fmoc Group

10 ml of deprotection (DEBLOCK) reagent was added to the resin and mixed well to react under gentle shaking for 5 min at room temperature. The solvent was discarded and the resin was washed 3 times with 10 ml of DMF for 2 min each time. The resin was washed 3 times with 6 ml of isopropanol for 5 min each time. The resin was washed 3 times with 6 ml of hexane for 5 min each time. The solvent was aspirated and discarded under vacuum. A small amount of resin sample was taken and the ninhydrin color method (Kaiser method) was used to quickly determine the free amino content on the resin: 2 ml of resin was washed with ethanol 3 times, 2 drops of 5% ninhydrin, 80% phenol and KCN (2 ml of 0.001 M KCN: 98 of ml piperidine) were added respectively, and were mixed well and heated at 120° C. for 4 to 6 min. The degree of deprotection reaction of the Fmoc group was determined.

### (4) Coupling Reaction of the Second Amino Acid

The connection of the second amino acid adopted the in situ activation method. 2 mmol of Fmoc amino acid, 4.0 mmol of TBTU and 4.0 mmol of HOBT were taken, to which a minimum amount of DMF was added to dissolve and 5 mmol of DIEA was added, and were mixed well and added to the Fmoc group-free resin. It was reacted under gentle shaking for 60 min at room temperature. The solvent was aspirated and discarded under vacuum. The resin was washed 3 times with 5 ml of methanol for 5 min each time. The resin was washed 3 times with 10 ml of DMF for 2 min each time. The solvent was aspirated and discarded under vacuum. A small amount of resin sample was taken for ninhydrin color analysis. The coupling rate was determined.

### (5) Extension Reaction of the Peptide Chain

The Fmoc protecting group at the N-terminus of the last amino acid was removed with 10 ml of DEBLOCK reagent, and the resin was washed 3 times with 10 ml of DMF, and the solvent was aspirated and discarded under vacuum. A small amount of resin sample was taken for ninhydrin color analysis. The next amino acid was coupled according to method (3). The deprotection of the Fmoc protecting group and amino acid coupling reaction were repeated until the desired polypeptide chain was obtained by coupling.

(6) Marking the N-Terminus of the Peptide Chain with Alexa647

The resin with all amino acid sequences was synthesized, the Fmoc protecting group at the N-terminus of the amino acid was removed, and the resin was washed 3 times with 10 ml of isopropyl alcohol for 5 min each time. 1.38 g of Alexa647, 1.6 g of TBTU53 and 0.76 ml of DIEA were mixed and added to the peptide-resin to react under gentle shaking for 60 min at room temperature. The solvent was aspirated and discarded under vacuum. The resin was washed 3 times with 5 ml of methanol for 5 min each time. The resin was washed 3 times with 10 ml of DMF for 2 min each time. The solvent was aspirated and discarded under vacuum.

(7) Deprotection of the Side Chain of the Peptide Chain and Cleavage from the Resin

The resin with all amino acid sequences synthesized was washed with 10 ml of DMF and then washed with 6 ml of isopropanol three times for 5 min each time. The resin was washed 3 times with 6 ml of hexane for 5 min each time. The solvent was aspirated and discarded under vacuum, and it was blown dry with N₂ and placed in a cracking vessel. 1 g of resin was added with 25 ml of cleavage reagent to react for 2 h at room temperature under shaking to mix well from time to time. After the reaction mixture was filtered through a glass filter to filter the resin, the cleavage reaction mixture was collected and the resin was washed 3 times with TFA. The reaction mixture was transferred to a round bottom flask, and washed 4 times with an equal volume of pre-chilled ether, and the precipitate was collected. After drying, the crude synthetic peptide was obtained.

### (8) Desalination of the Synthetic Peptide

The crude peptide was dissolved in distilled water. 15 g of Amersham G-25 gel was weighed, swelled and installed into the column. The packed column was equilibrated with 50 ml of distilled water, loaded with 3 to 5 ml of sample each time after equilibration, and eluted with distilled water. The ultraviolet spectrophotometer was used to detect the ultraviolet absorption at 220 nm and the peptide was collected according to the peak.

(9) Purifying the Peptide with HPLC

The peptide was separated and purified using Waters Delta Prep 4000 preparative HPLC high performance chromatograph from Waters. The column was a radial pressurized column (25×100, 15 μm, DELTA PAKC18 packing), and the elution system was: liquid A: 5% acetonitrile solution (containing 0.1% TFA); liquid B: 95% acetonitrile solution (containing 0.08% TFA). Manual injection with 1 ml per injection, flow rate of 4 ml/min, and linear gradient, within 45 min, liquid B was rose from 5% to 50%, and then rose to 95% liquid B within 5 min for final elution. The UV absorption is detected at 215 nm, and the components were collected according to the peaks for mass spectrometry detection. The components with correct molecular weight detection were collected and lyophilized in vacuum to become the pure product required and set aside.

### Example 2 Localization of the pH Low Insertion Peptide on Tumor Cells Cultured In Vitro

### 1. Cell Line

Human colorectal cancer cell line SW480 (purchased from ADCC).

### 2. Reagents

RPMI 1640 medium (solarbio), fetal bovine serum (Yuanheng Jinma Corporation), PBS (pH=7.4) (Gibco), hydrochloric acid, alexa647 marked var7 (the var7 is a standard var7, and the sequence is: Ala-Cys-Glu-Glu-Gln-Asn-Pro-Trp-Ala-Arg-Tyr-Leu-Glu-Trp-Leu-Phe-Pro-Thr-Gl u-Thr-Leu-Leu-Leu-Glu-Leu (SEQ ID NO. 8)) and alexa647 marked p-var7 (the p-var7 is a lengthened version of the var7 with the extracellular segment of the var7 repeated once, and the sequence is: Ala-Cys-Glu-Glu-Gln-Asn-Pro-Gly-Gly-Gly-Ser-Ala-Cys-Glu-Glu-Gln-Asn-Pro-Trp-Ala-Arg-Tyr-Leu-Glu-Trp-Leu-Phe-Pro-Thr-Glu-Thr-Leu-Leu-Leu-Glu-Leu (SEQ ID NO. 18)), and alexa647 is attached to the Cys at the N-terminal position 2 of the above two polypeptides.

### 3. Instruments

Ultra clean table (RONGFENG), carbon dioxide incubator (Thermo), centrifuge (Thermo), laser confocal cell culture dish (20 mm) (Corning), electronic pH meter (Satoris), optical microscope (Olympus), laser confocal microscope (Nikon).

### 4. Experimental Method

(1) The SW480 cells in logarithmic phase were collected, the culture solution was discarded, and the cells were washed twice with saline. Appropriate amount of 0.25% trypsin was added to digest until the cells did not adhere to the wall, and appropriate amount of culture solution was added to stop the digestion. The cells were transferred to a 10 ml test tube to centrifuge at 1000 rpm for 5 min, the supernatant was removed, and 1 ml of RPMI 1640 medium containing 10% fetal bovine serum was added to resuspend and mix the cells. After 10 μl of the cell suspension was added to a cell counting plate and counted, a certain amount of the cell suspension was added into the laser confocal cell culture dish, which was then adjusted to a cell system of 5*10⁵, 1 ml with complete medium, and put into a cell incubator and incubated overnight.

(2) The culture solution was prepared using 1 mol/L hydrochloric acid and a PBS buffer at pH 7.4. Hydrochloric acid was added dropwise to the PBS buffer, and finally the pH value of the buffer was titrated to 6.3. The synthesized peptides (p-var7 and var7) were added to PBS buffers at pH 6.3 and 7.4, respectively, and mixed well, and diluted proportionally to a final peptide concentration of 2.5 μmol/L, which was the peptide-containing PBS culture solution.

(3) When the SW480 cells adhered after being cultured overnight, the supernatant of the medium was removed, and the cells were washed twice with a PBS buffer at pH 7.4.

(4) The PBS buffer in the cell culture dish was removed, 1 ml of the previously prepared mixed culture buffers of PBS at pH 6.3 and 7.4 and peptides were added to the two dishes, respectively, and 1 ml of the PBS medium at pH 7.4 without peptides was added to another control dish, and all of them were put in a 37° C. cell incubator to incubate for 1 hour.

(5) After incubation, the supernatant of the peptide-containing PBS culture solution was removed, and the peptide-containing PBS culture solution was washed three times with peptide-free PBS buffers with different pH values in each group, and then added with a PBS buffer at pH 7.4.

(6) The prepared cell culture dishes were placed under the laser confocal microscope (647 mm excitation wavelength) to observe the fluorescence expression on the surface of the cell membrane.

### 3. Experimental Results:

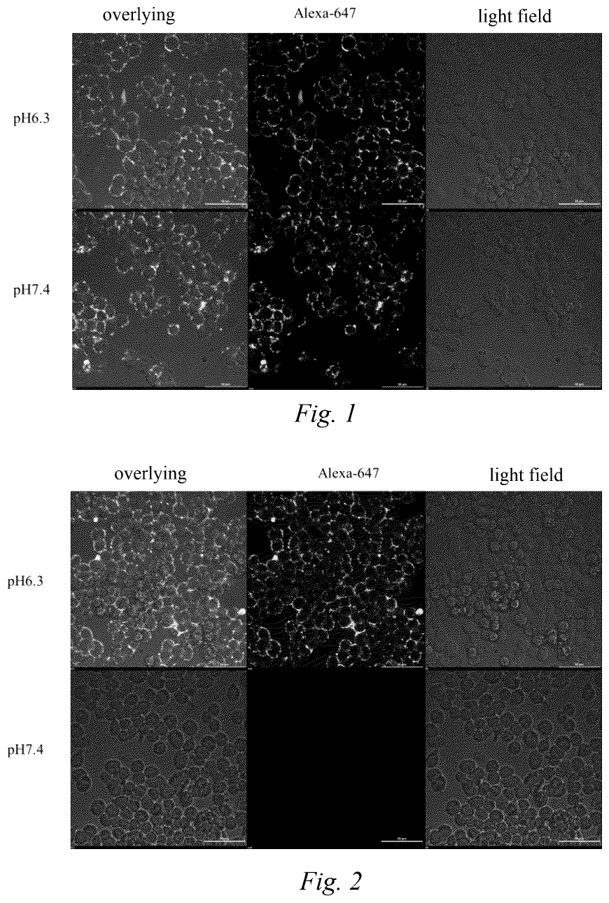

The results were shown in FIG. 1 and FIG. 2, both the var7 and the p-var7 could effectively insert into the surface of human colon cancer cell SW480 in an acid solution environment, but the membrane insertion ability of the p-var7 was lost in a neutral solution environment (FIG. 2), while the var7 retained the ability (FIG. 2), showing that the p-var7 was more selective in the acidic microenvironment of tumor tissues.

### Example 3 Localization of the pH Low Insertion Peptide in Animals

### 1. Experimental Steps:

Mouse colon cancer cells CT26 were subcutaneously inoculated into Balb/c mice, and when the tumors grew to about 1 cm, saline (N.S.), alexa647 marked p-var7, alexa647 marked var7 were intravenously injected respectively with a dosage of 60 μM/100 μL N.S., in vivo imaging (excitation with Cy5.5 wavelength) was performed at 24, 48, 72 hours with supine position photography. The mice were sacrificed for tumor imaging.

### 2. Experimental Results

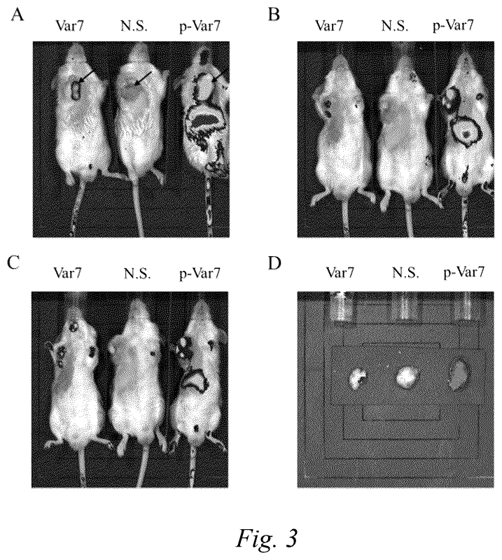

The results shown in FIG. 3 showed that both the var7 and the p-var7 could mark tumors, but the p-var7 had stronger marking ability, and as time went by, the fluorescence intensity of the var7 decayed more significantly, and by 72 hours, only a few markers were visible in the tumor tissues, on the contrary, the p-var7 still showed strong fluorescent marking by 72 hours. This experiment proved that the p-var7 could be targeted to tumor tissues in vivo and maintained for a longer period of time. The black arrow in Figure A represented tumor tissues.

### Example 4 Evaluation of the Anti-Tumor Effect of the Antibodies Prepared Using the Extracellular Domain of the p-Var7 as the Antigen

Experimental Materials: MC38 cells, purchased from ATCC; p-var7 polypeptide (molecular weight is 4095 Da), synthesized by Beijing Huada Protein R & D Center Co., Ltd and dissolved in PBS at a concentration of 40 μM; extracellular domain of the p-var7 (Ala-Cys-Glu-Glu-Gln-Asn-Pro-Gly-Gly-Gly-Ser-Ala-Cys-Glu-Glu-Gln-Asn-Pro, SEQ ID NO. 22) connected to KLH (synthesized by Beijing Genscript Biotechnology Co., Ltd); 6 to 8 week old female C57/BL6 mice purchased from Vital River.

### 1. Preparation of the Antibodies:

(1) Step: Balb/c mice were immunized with the extracellular domain of the p-var7 connected by KLH to prepare hybridomas, and two monoclonal antibodies were obtained, and the specific binding of antibody to antigen and the antibody subtype were detected by ELISA.

(2) Results:

The results showed that the monoclonal antibodies named 1G12 and 1G1 could specifically bind to the antigen, wherein the affinity of 1G12 was higher, the affinity of 1G1 was lower, the OD values were 1.4423 and 0.4924 respectively, and both the two strains were of IgG1 subtype, and the concentration of the prepared antibodies was about 0.7 mg/ml.

### 2. Evaluation of the Anti-Tumor Effect of the Antibodies

(1) Establishment of MC38 transplanted tumor model of mouse colon cancer: MC38 was inoculated into C57/BL6 mice, when the tumor diameter grew to 1 cm, the tumor was aseptically peeled off, shredded, homogenized, and sieved to make a single cell suspension which was cultured and amplified in a 1640 complete medium. The cells were injected into the subcutaneous abdomen of the C57/BL6 mice with 2×10⁶ cells/body, when the tumor diameter grew to 0.8 to 1 cm, the oversized and undersized tumors were removed, and mice with substantially the same tumor size were grouped. There were 4 groups: injection group of the p-var7 alone, 10; injection group of the p-var7 combined with the 1G12, 10; injection group of the p-var7 combined with the 1G1, 10; injection group of normal saline N.S, 10. The tumor size was measured every 3 days.

(2) Administration method: p-var7 administration: each was injected intravenously with 40 μM/100 μl (approximately 16 μg, referring to the previous experiment results of in vivo imaging, the accumulation of fluorescence in the tumor sites could be observed with the same dose of injection, but it basically degraded by the third day) every time, starting from the day after the grouping was completed, once a day, and once every 2 days; antibody administration: intraperitoneal injection, the dose was 5 mg/kg (the molar ratio of the antibody to the p-var7 was about 1:5), the injection frequency was the same as the p-var7, and the injection time was 6 to 12 hours after the p-var7 administration until the end.

(3) Results:

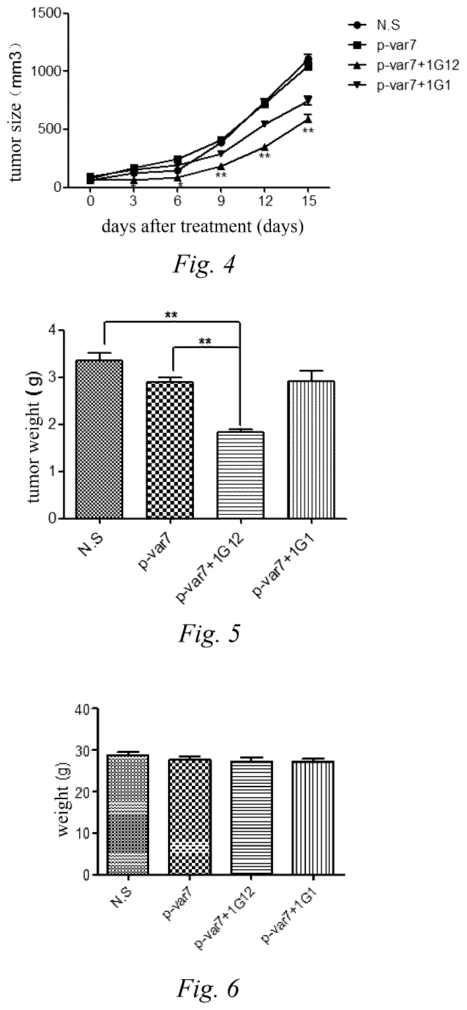

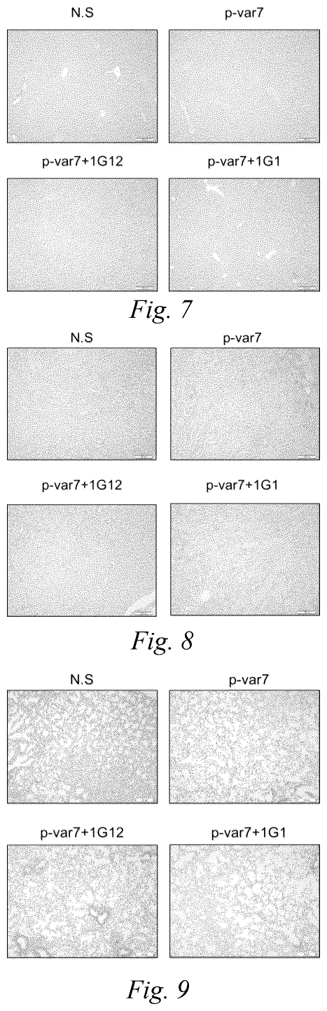

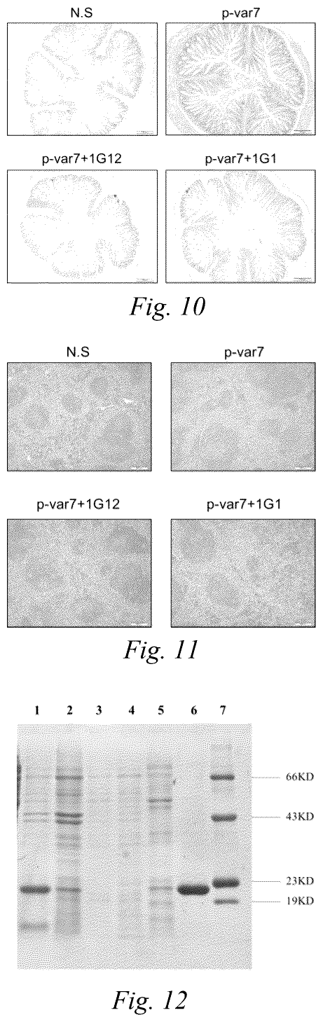

The results were shown in FIG. 4 and FIG. 5, the 1G12 could significantly inhibit tumor growth, the inhibition rate was 50% at 2 weeks, and the 1G1 had no obvious tumor suppressing effect; during the treatment, the mice were in good health, and there were no symptoms such as decreased activity, diarrhea, and weight loss, and FIG. 6 showed that the body weight of the mice remained unchanged; pathological results showed that the organs of the liver, kidney, lung, spleen and intestine in the treatment group had no organic changes (FIGS. 7-11).

### Example 5 Synthesis of the Fourth Domain of the Her2-pHLIP

### 1. Prokaryotic Expression

### Step:

### Strain Construction

The DNA sequence was designed according to the amino acid sequence as shown in SEQ ID NO. 21 for complete gene synthesis, and the restriction sites Nde I and XhoI sequences were connected to the two ends during the synthesis, and the fourth domain of the her2-pHLIP encoding nucleic acid and the PET28a vector were digested with the two enzymes respectively. The digested fragments and vectors were recovered, ligated with the T4 ligase, transformed into the recipient strain BL21 (DE3), plated and cultured, and clones were picked for sequencing.

### Culture Induction

The clones with the correct sequencing were cultured (LB medium, 37° C.). When the OD value reached 0.6 to 0.8, IPTG was used for induction, the final concentration of IPTG was 0.5 mM, and the bacteria were collected by centrifugation for 4 to 6 hours.

### 2. Purification of the Inclusion Body

### Step:

### (1) Washing of the Inclusion Body

Liquid A: 50 mM Tris, 2 mM EDTA, pH 8.0, washed twice;
Liquid B: 50 mM Tris, 2 mM EDTA, 0.1% Triton, pH 8.0, washed once;
Liquid C: 20 mM Tris, 1M urea, pH 8.0, washed once.

### (2) Renaturation and Nickel Column Purification

Extraction: The inclusion body was extracted with 8 M urea, 5 mM β-Me, 0.3 M NaCl, 20 mM Tris, pH 8.0 with an extraction ratio of 1:20.

Renaturation dialysis: The target protein was supplemented with 10 mM β-Me to be reduced at 40° C. for 15 minutes and diluted to 0.2 mg/ml. The sample was dialyzed against 10 mM PB and 50 μM CuCl₂, pH 8.0, the dialyzate was changed 3 times, and the supernatant was collected by centrifugation.

Ni column purification The column was equilibrated with 0.3 M NaCl, 10 mM PB, pH 8.0, and washed with an equilibration buffer containing 40 mM imidazole after loading, and the target protein was eluted with an equilibration buffer containing 300 mM imidazole. The purity of the target protein was greater than 95%.

Desalination: Desalted into 20 mM PB, 0.1 M NaCl.

### (3) SDS-PAGE

The sample processed in the step (2) was subjected to SDS-PAGE. The results shown in FIG. 12 showed that the experiment could isolate and purify the composition of the present invention. Note: in FIG. 12, lane 1: GJ inclusion body renaturation; lane 2: sample flow lanes 3 and 4: Buffer liquid balance; lane 5: 40 mM imidazole elution; lane 6: 300 mM imidazole elution; lane 7: Marker.

### Example 6 Localization of the Fourth Domain of the Her2-pHLIP on Tumor Cells Cultured In Vitro

### 1. Cell Culture

A549 cells were cultured with a DMEM medium containing 10% calf serum and 1.6 million units of gentamicin/ml in a cell incubator with at 37° C. and 5% CO₂. After the cells were overgrown, they were passaged at 1:10.

### 2. Confocal Observation Localization

A549 cells (5×10⁵/well) were cultured on coverslip dishes overnight, the culture supernatant was discarded, PBS at pH 6.3 and 7.4 were added respectively, and the fourth domain of the her2 pHLIP (60 μg/ml) expressed in the example 5 was added to incubate at 37° C. for 1 hour. The supernatant was discarded and PBS of corresponding pH was used to wash 3 times, PBS at pH 6.3 and 7.4 were added, and a trastuzumab-FITC or TGLA-FITC (control antibody) (the concentration was 1:400 dilution) was added to incubate at 37° C. for 30 min. The supernatant was discarded and PBS of corresponding pH was used to wash 3 times, PBS at pH 7.4 was added and confocal observation. Grouping: (1) pH 6.3 untreated group; (2) pH 6.3 composition (the fourth domain of the her2-pHLIP); (3) pH 6.3 composition (the fourth domain of the her2-pHLIP)+trastuzumab-FITC; (4) pH 6.3 composition (her2 fourth domain-pHLIP)+TGLA-FITC; (5) pH 7.4 composition (the fourth domain of the her2-pHLIP)+trastuzumab-FITC; (6) pH 7.4 trastuzumab-FITC.

### 3. Results

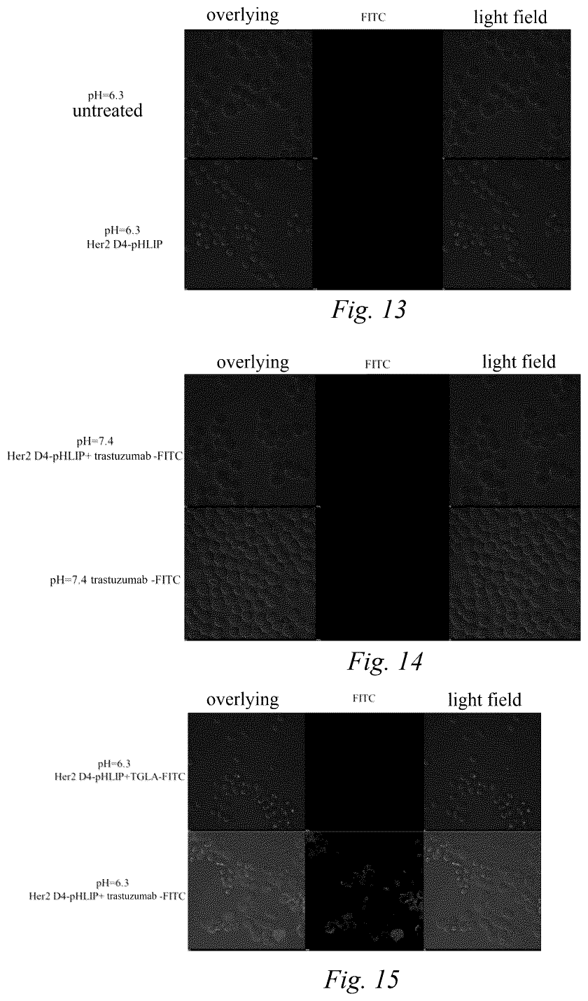

FIG. 13 showed that the human lung cancer cell line A549 did not express the Her2.

FIG. 14 showed that in a neutral solution environment, the composition of the present invention could not insert into the cell membrane of the A549 and could not display the fourth domain of the Her2 on the cell membrane.

FIG. 15 showed that in an acidic solution environment, the composition of the present invention could insert into the cell membrane of the A549, and the fourth domain of the Her2 displayed on the cell membrane could be recognized by the trastuzumab.

The above results indicated that the connection of the pH low insertion peptide to the fourth domain of the Her2 did not affect its property of the pH low insertion peptide insertion into the cell membrane, while the connection of the fourth domain of the Her2 to the pH low insertion peptide did not affect its conformation.

### Example 7 Evaluation of the Effect of the Fourth Domain of the Her2-pHLIP and Herceptin on Tumor Treatment

Experimental Materials: A549 cells, purchased from ATCC; prokaryotic expression of the fourth domain of the Her2-pHLIP (Her2 D4-pHLIP); Herceptin was purchased from Roche Pharmaceuticals; 6 to 8 week old male nude mice were purchased from Vital River.

### Experimental Steps:

A549 was inoculated into nude mice, when the tumor diameter grew to 1 cm, the tumor was aseptically peeled off, shredded, homogenized, and sieved to make a single cell suspension which was cultured and amplified in 1640 complete medium. The cells were injected into the subcutaneous abdomen of the nude mice with 1×10⁶ cells/body, when the tumor diameter grew to 0.5 to 1 cm, the oversized and undersized tumors were removed, and mice with substantially the same tumor size were grouped. There were 4 groups: injection group of the Her2 D4-pHLIP alone, 10; injection group of the Her2 D4-pHLIP combined with the herceptin, 10; injection group of the Her2 D4-pHLIP combined with the IgG1, 10; injection group of normal saline N.S, 10. The tumor size was measured every 3 days.

Administration method: Her2 D4-pHLIP administration: each was injected intravenously with 40 μM/100 μl every time, starting from the day after the grouping was completed, once a day, and once every 2 days; antibody administration: Intraperitoneal injection, the dose was 10 mg/kg, the injection frequency was the same as the Her2 D4-pHLIP, and the injection time was 6 to 12 hours after the administration of the Her2 D4-pHLIP until the end.

### Result Analysis:

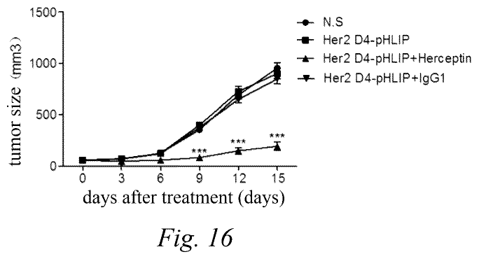

The results in FIG. 16 showed that the Herceptin significantly inhibited tumor growth in lung cancer mice.

The above contents are further detailed descriptions made for the present invention in conjunction with particular preferred embodiments, and it should not be interpreted that the particular embodiments of the present invention are only limited to these descriptions. For a person of ordinary skill in the art, various simple deductions and substitutes can be made without departing from the concept of the present invention, which should be considered as falling within the scope of protection of the present invention.

1. An improved pH low insertion peptide, wherein the improved pH low insertion peptide comprises the following sequences: sequences obtained by repeating the extracellular domain of the pH low insertion peptide having the sequence of SEQ ID NO. 1 or a variant thereof once, twice or more times; and preferably, the variant of the pH low insertion peptide having the sequence of SEQ ID NO. 1 comprises polypeptides having the sequences as shown in SEQ ID NO. 2 to SEQ ID NO. 17.

2. The improved pH low insertion peptide according to claim 1, wherein the sequence of the improved pH low insertion peptide from N-terminus to C-terminus is shown as follows: (extracellular domain) n+linker+the pH low insertion peptide having the sequence of SEQ ID NO. 1 or the variant thereof, where n=1, 2, 3, 4 . . . ; preferably, the sequence of the linker is (GGGS) m, where m=1, 2, 3, 4 . . . ; and more preferably, the sequence of the linker is GGGS.

3. The improved pH low insertion peptide according to claim 1, wherein the sequence of the improved pH low insertion peptide is shown in SEQ ID NO. 18.

4. A composition, wherein the composition comprises the improved pH low insertion peptide of claim 1.

5. The composition according to claim 4, wherein the composition comprises a functional body which comprises therapeutic agents, diagnostic agents, and marker molecules; the functional body is connected to the N-terminus or C-terminus of the improved pH low insertion peptide, or to the N-terminus or C-terminus of the pH low insertion peptide having the sequence of SEQ ID NO. 1 or the variant thereof.

6. The composition according to claim 5, wherein the therapeutic agent comprises antibody drugs, small molecule drugs, antibiotics, polypeptides, peptide nucleic acids, nanoparticles, or liposomes; preferably, the polypeptide comprises toxins, cyclic peptides, microtubule inhibitors, protease activated receptors; and preferably, the peptide nucleic acid comprises antisense nucleic acid oligonucleotide peptides.

7. The composition according to claim 5, wherein the diagnostic agents comprise fluorescent dyes.

8. The composition according to claim 5, wherein the marker molecules comprise tumor surface antigens or functional domains thereof; the functional domains of the tumor surface antigens are domains recognizing and binding to the antibody against the tumor surface antigens; and preferably, the tumor surface antigens comprise ER, PR, P53, EGFR, IGFR, Her2, CD20, CD25, CD117, CD34, CD138, CD33, VEGFR, BCMA, Mesothelin, CEA, PSCA, MUC1, EpCAM, S100, CD22, CD19, CD70, CD30, ALK, RANK, GPC2, GPC3, Her3, EGFRvIII, GD2, PD-L1, PD-L2.

9. The composition according to claim 8, wherein the marker molecules are connected to the N-terminus of the improved pH low insertion peptide, or to the N-terminus of the pH low insertion peptide having the sequence of SEQ ID NO. 1 or the variant thereof via a linker; preferably, the sequence of the linker is (GGGS) m or (GGGGS) m, where m=natural number; and more preferably, the sequence of the linker is GGGGS.

10. The composition according to claim 9, wherein the marker molecules are connected to the N-terminus of the pH low insertion peptide having the sequence of SEQ ID NO. 1 via a linker, preferably, the sequence of the linker is (GGGS) m or (GGGGS) m, where m=natural number; and more preferably, the sequence of the linker is GGGGS.

11. The composition according to claim 8, wherein the marker molecules are tumor surface antigen Her2 or functional domains thereof.

12. The composition according to claim 11, wherein the functional domain of the Her2 is a fourth domain of an Her2 protein or a functionally similar domain thereof, or a second domain of the Her2 protein or a functionally similar domain thereof, the sequence of the fourth domain of the Her2 protein is shown in SEQ ID NO. 20, the functionally similar domain of the fourth domain of the Her2 protein is a polypeptide derived from the amino acid as shown in SEQ ID NO. 20 and retaining the antibody binding activity of the fourth domain of the Her2 protein, and the sequence of the second domain of the Her2 protein is shown in SEQ ID NO. 23, and the functionally similar domain of the second domain of the Her2 protein is a polypeptide derived from the amino acid as shown in SEQ ID NO. 23 and retaining the antibody binding activity of the second domain of the Her2 protein.

13. The composition according to claim 12, wherein the functional domain of the Her2 is the fourth domain of the Her2 protein or the second domain of the Her2 protein.

14. The composition according to claim 13, wherein the fourth domain of the Her2 protein is connected to the N-terminus of the pH low insertion peptide having the sequence of SEQ ID NO. 1 via a linker, the sequence of the linker is GGGGS, and the sequence of the composition is shown in SEQ ID NO. 21; and the second domain of the Her2 protein is connected to the N-terminus of the pH low insertion peptide having the sequence of SEQ ID NO. 1 via a linker, the sequence of the linker is GGGGS, and the sequence of the composition is shown in SEQ ID NO. 24.

15. A neoantigen, wherein the neoantigen sequence comprises the extracellular domain sequence of the improved pH low insertion peptide of claim 1 or a variant sequence thereof, or the extracellular domain sequence of the improved pH low insertion peptide shown in SEQ ID NO. 1.

16. A nucleic acid molecule, wherein the nucleic acid molecule encodes the neoantigen of claim 15.

17. A fusion protein, wherein the fusion protein comprises the neoantigen of claim 15 and a protein or polypeptide connected to the neoantigen.

18. A novel antibody, wherein the novel antibody is prepared from the neoantigen of claim 15.

19. A tumor marking system, wherein the tumor marking system comprises the improved pH low insertion peptide of claim 1.

20. The tumor marking system according to claim 19, wherein the tumor marking system comprises the composition of claim 8.

21. A targeted tumor therapeutic system, wherein the targeted tumor therapeutic system comprises the tumor marking system of claim 19, the tumor marking system of claim 19 and a tumor killing system which comprises the novel antibody of claim 18.

22.-26. (canceled)


SEQUENCE LISTING (24 sequences)

SEQ ID NO: 1 - protein, 36 aa, Homo sapiens
ACEQNPIYWARYADWLFTTPLLLLDLALLVDADEGT

SEQ ID NO: 2 - protein, 32 aa, Homo sapiens
ACEDQNPYWARYADWLFTTPLLLLDLALLVDG

SEQ ID NO: 3 - protein, 30 aa, Homo sapiens
ACEDQNPYWRAYADLFTPLTLLDLLALWDG

SEQ ID NO: 4 - protein, 27 aa, Homo sapiens
ACDDQNPWRAYLDLLFPTDTLLLDLLW

SEQ ID NO: 5 - protein, 27 aa, Homo sapiens
ACEEQNPWRAYLELLFPTETLLLELLW

SEQ ID NO: 6 - protein, 25 aa, Homo sapiens
ACDDQNPWARYLDWLFPTDTLLLDL

SEQ ID NO: 7 - protein, 24 aa, Homo sapiens
CDNNNPWRAYLDLLFPTDTLLLDW

SEQ ID NO: 8 - protein, 25 aa, Homo sapiens
ACEEQNPWARYLEWLFPTETLLLEL

SEQ ID NO: 9 - protein, 24 aa, Homo sapiens
CEEQQPWAQYLELLFPTETLLLEW

SEQ ID NO: 10 - protein, 24 aa, Homo sapiens
CEEQQPWRAYLELLFPTETLLLEW

SEQ ID NO: 11 - protein, 24 aa, Homo sapiens
ACEDQNPWARYADWLFPTTLLLLD

SEQ ID NO: 12 - protein, 24 aa, Homo sapiens
ACEEQNPWARYAEWLFPTTLLLLE

SEQ ID NO: 13 - protein, 22 aa, Homo sapiens
ACEDQNPWARYADLLFPTTLAW

SEQ ID NO: 14 - protein, 22 aa, Homo sapiens
ACEEQNPWARYAELLFPTTLAW

SEQ ID NO: 15 - protein, 34 aa, Homo sapiens
TEDADVLLALDLLLLPTTFLWDAYRAWYPNQECA

SEQ ID NO: 16 - protein, 37 aa, Homo sapiens
CDDDDDNPNYWARYANWLFTTPLLLLNGALLVEAEET

SEQ ID NO: 17 - protein, 37 aa, Homo sapiens
CDDDDDNPNYWARYAPWLFTTPLLLLPGALLVEAEET

SEQ ID NO: 18 - protein, 36 aa, Artificial Sequence
  Fusion protein
ACEEQNPGGGSACEEQNPWARYLEWLFPTETLLLEL

SEQ ID NO: 19 - protein, 5 aa, Artificial Sequence
  protein segment
GGGGS

SEQ ID NO: 20 - protein, 138 aa, Artificial Sequence
  Fusion protein
HHHHHHPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEE

SEQ ID NO: 21 - protein, 178 aa, Artificial Sequence
  Fusion protein
HHHHHHPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGGGGSACEQNPIYWARYADWLFTTPLLLLDLALLVDADEG

SEQ ID NO: 22 - protein, 18 aa, Artificial Sequence
  Fusion peptide
ACEEQNPGGGSACEEQNP

SEQ ID NO: 23 - protein, 156 aa, Artificial Sequence
  protein segment
HHHHHHILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCP

SEQ ID NO: 24 - protein, 196 aa, Artificial Sequence
  protein segment
HHHHHHILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPGGGGSACEQNPIYWARYADWLFTTPLLLLDLALLVDADEG